(19) United States
(12) Patent Application Publication
(10) Pub. No.: US 2023/0293904 A1
(43) Pub. Date: Sep. 21, 2023

(54) MAGNETIC MUSCLE STIMULATOR WITH MICROCHANNEL COOLING SYSTEM

(71) Applicant: Yassen Wellness LLC, Irvine, CA (US)

(72) Inventors: Ewan Yassen, Irvine, CA (US); Mahdi Yassen, Irvine, CA (US)

(73) Assignee: Yassen Wellness LLC, Irvine, CA (US)

(21) Appl. No.: 18/186,162

(22) Filed: Mar. 18, 2023

Related U.S. Application Data
(60) Provisional application No. 63/321,603, filed on Mar. 18, 2022.

(51) Int. Cl. A61N 2/02; A61N 2/00

(52) U.S. Cl. CPC A61N 2/02; A61N 2/004

(57) ABSTRACT

A magnetic muscle stimulator device comprises a water tank configured to hold a quantity of water, a pump, one or more fans, and a magnetic applicator comprising a coil. The coil generates a magnetic field in response to receiving an alternating current. The coil comprises a water passage in the interior of the coil. The pump receives water from the water tank through a first water pipe and transfers the water into the water passage of the coil through a second water pipe. The water absorbs heat from the coil while passing through the water passage of the coil. The pump receives the water from the water passage of the coil through a third pipe and returns the water into the water tank through a fourth pipe. The water is cooled by the fans while being returned from the water passage of the coil into the water tank.

### CLAIM OF BENEFIT TO PRIOR APPLICATIONS

This application claims the benefit of U.S. Provisional Pat. Application Serial No. 63/321,603, filed on Mar. 18, 2022. The contents of U.S. Provisional Pat. Application 63/321,603 are hereby incorporated by reference.

### BACKGROUND

Non-invasive devices for aesthetic treatments include devices that apply magnetic fields on a person’s muscles to reduce body fat, provide pain relief, or provide muscle relaxation. The magnetic fields may be applied by a magnetic applicator that includes a coil and is connected to a power source.

### BRIEF DESCRIPTION OF THE DRAWINGS

The various embodiments of the present magnetic muscle stimulator with microchannel cooling system now will be discussed in detail with an emphasis on highlighting the advantageous features. These embodiments depict the novel and non-obvious magnetic muscle stimulator with microchannel cooling system shown in the accompanying drawings, which are for illustrative purposes only. These drawings include thefollowing FIGS., in which like numerals indicate like parts:

FIG. 1 is a functional block diagram illustrating an example magnetic muscle stimulator with a water cooling system, according to various aspects of the present disclosure;

FIG. 2 is a top perspective view of a cross section of a magnetic applicator, according to various aspects of the present disclosure;

FIGS. 3A and 3B illustrate cross sections of coils 140 of magnetic applicators that include a microchannel for passing cooling water through, according to various aspects of the present embodiments;

FIG. 4 illustrates a cross section of a coil of a magnetic applicator that includes several microchannels for passing cooling water through, according to various aspects of the present embodiments;

FIG. 5 is a front perspective view of a magnetic muscle stimulator, according to various aspects of the present disclosure;

FIG. 6 is a back perspective view of a magnetic muscle stimulator, according to various aspects of the present disclosure;

FIG. 7 is a front perspective view of a magnetic applicator connector that connects the magnetic applicator connector to a matching connector on the housing of the magnetic muscle stimulator, according to various aspects of the present disclosure;

FIG. 8 is a front perspective view of a connector on the housing of the magnetic muscle stimulator that connects to a matching connector on a magnetic applicator connector, according to various aspects of the present disclosure;

FIG. 9 is a schematic front view of the display of a magnetic muscle stimulator with a user interface that indicates the working time of each magnetic applicator, according to various aspects of the present disclosure;

FIG. 10 is a schematic front view of the display of a magnetic muscle stimulator with a user interface that provides a warning when a magnetic applicator exceeds the maximum working time, according to various aspects of the present disclosure;

FIG. 11 is a schematic front view of the display of a magnetic muscle stimulator with a user interface that provides a warning when the magnetic muscle stimulator has insufficient water flow, according to various aspects of the present disclosure; and

FIG. 12 conceptually illustrates an electronic system 1200 with which some embodiments of the invention (e.g., the magnetic muscle stimulator 100, described above) are implemented.

### DETAILED DESCRIPTION

One aspect of the present embodiments includes the realization that the magnetic muscle stimulators include a magnetic applicator with a coil that heats up during use. The magnetic applicator requires a cooling system to prevent injury to the patient and prolog the use of the magnetic applicator. The cooling system of the existing magnetic muscle stimulators suffer from being incapable of adequately distributing the cooling fluid throughout the magnetic applicator. As a result, the magnetic applicator has to be switched off frequently to cool down.

The present embodiments, as described in detail below, solve the above-mentioned problems by providing a cooling system that circulates water through the magnetic applicator. The magnetic applicator of the present embodiments includes one or more microchannel passages inside the heated coil through which the cooling water is circulated.

The remaining detailed description describes the present embodiments with reference to the drawings. In the drawings, reference numbers label elements of the present embodiments. These reference numbers are reproduced below in connection with the discussion of the corresponding drawing features.

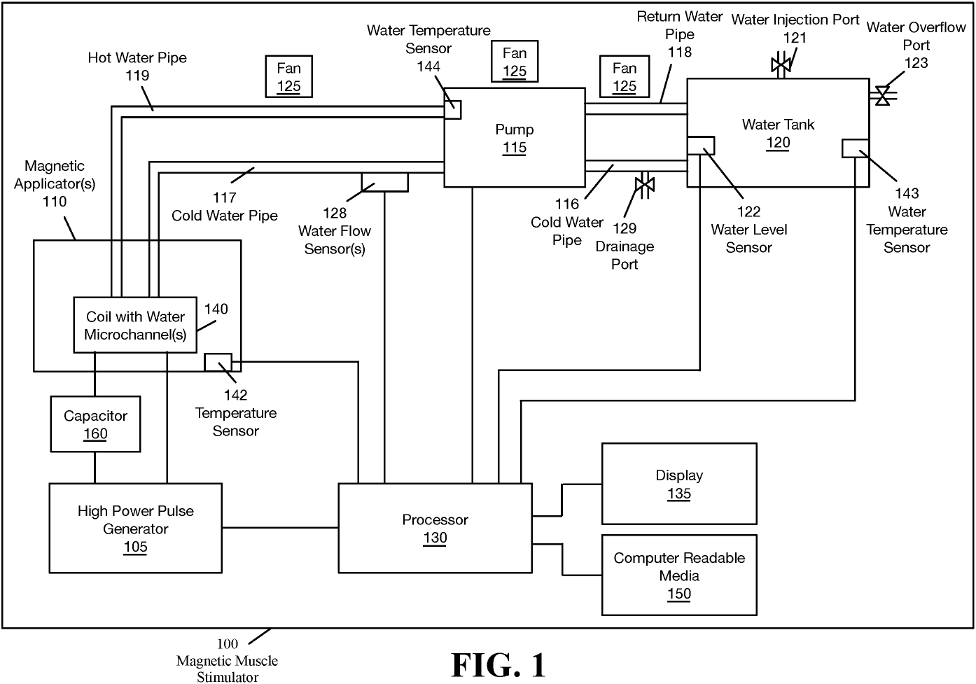

Some of the present embodiments provide a novel cooling system for the magnetic applicator of a muscle stimulators. FIG. 1 is a functional block diagram illustrating an example magnetic muscle stimulator with a water cooling system, according to various aspects of the present disclosure. With reference to FIG. 1, the magnetic muscle stimulator 100 may include one or more high power pulse generators 105, one or more magnetic applicators 110, one or more capacitors 160, a pump 115, a water tank 120, one or more fans 125, a processor 130, computer readable media 150 (e.g., volatile memory and non-volatile memory), and a display 135.

The computer readable media 150 may be non-transitory computer readable media. The computer readable media 150 may include different types of memory units, such as, read-only-memory, volatile read-and-write memory, and/or non-volatile read-and-write memory. The read-only-memory may store static data and instructions that are needed by the processor 130. The non-volatile read-and-write memory may store instructions and data even when the power to the non-volatile memory is off. Some embodiments may use a mass-storage device (such as a magnetic or optical disk and its corresponding disk drive) as the non-volatile read-and-write memory.

The volatile read-and-write memory device may be random access memory and may be used as system memory. The system memory may store some of the instructions and data that the processor needs at runtime. In some embodiments, the processes of the present embodiments may be stored in the system memory, the non-volatile memory, and/or the read-only memory. From these various memory units, the processor 130 may retrieve instructions to execute and data to process in order to execute the processes of some embodiments.

The magnetic muscle stimulator 100 produces an electromagnetic field that interacts with the tissues of the human body. The main affected tissue structures are muscular, collagenous, and neuronal tissues. The magnetic muscle stimulator 100 is a non-invasive device and the electromagnetic field may be delivered by the magnetic applicator(s) 110 in the subdermal, muscular or collagenous tissue area triggering the stimulation and relaxation.

The magnetic muscle stimulator 100 may include one or more magnetic applicators 110. Each magnetic applicator 110 may correspond to a high power pulse generator 105 and a capacitor 160 (only one magnetic applicator 100, and the corresponding high power pulse generator 105 and capacitor 160 are shown for clarity).

The high power pulse generator(s) 105 may receive power from an external power source (e.g., from an alternating current (AC) outlet) and may generate high power AC, for example, in the form of electrical pulses. The high power pulse generator(s) 105, in some embodiments, may work with AC power sources ranging from 100-240 volts (V) AC, 50-60 hertz (Hz), and a current of up to 14 amperes (A). The AC power, in some embodiments, may be filtered through a power filter (not shown) to remove the undesirable harmonics. The high power pulse generator(s) 105, in some embodiments, may produces electrical pulses with a power ranging between 1,200 volt-amperes (VA) to 2,300 VA. The capacitor 160 may be charged and discharged by the corresponding high power pulse generator 105 and may provide energy in the form of AC current to the corresponding coil 140. The capacitor 160, in some embodiments, may be in the range of 100 microfarad (uF) to 130 uF. The coil 140 and the corresponding high power pulse generator 105 and capacitor 160 may be connected in series or in parallel in different embodiments.

During therapy, the magnetic applicator(s) 110 may be held by hand (e.g., by an operator of the magnetic applicator 100) or may be fixed to a person’s body by one or more straps. Each magnetic applicator 110 may include a coil 140. The coil 140 may receive the high power electrical pulses from the high power pulse generator 105, and may generate a controllable magnetic field. When the high power electrical pulses from the high power pulse generator 105 are turned off, the coil 140 no longer generates the magnetic field.

The processor 130 may display a user interface on the display 135. A person (e.g., the operator of the magnetic muscle stimulator 100) may select different therapy programs, may change different parameters of a therapy program, and/or may monitor the operations of the magnetic muscle stimulator 100 through the user interface.

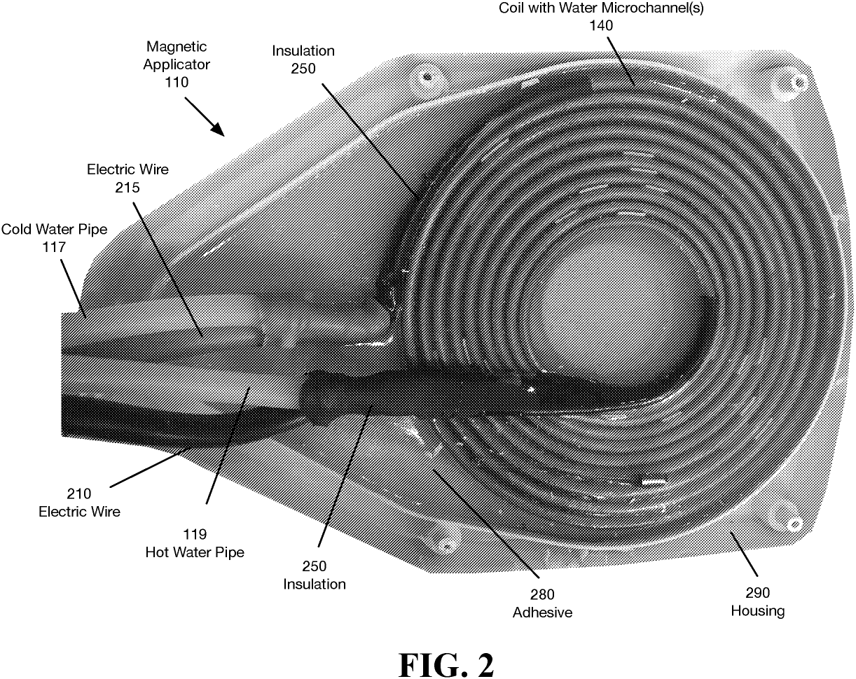

The coil 140 may generate a magnetic field strength (or magnetic flux density) that may controllably change from 0.5 tesla (T) to 13.5 T, depending on the therapy program selected through the user interface and the type of the magnetic applicator 110 (one tesla is equal to 10,000 gauss (G)). The coil 140, in some embodiments, may generate a magnetic frequency that may controllably change between 1-150 Hz. As an example, an average working frequency of 16-17 Hz may get over 30,000 contractions for the muscle in 30 minutes (min). The magnetic applicator 110 may include a housing 290 (shown in FIG. 2) that may encompass the coil 140 to prevent the coil 140 from touching a person’s body.

The magnetic field, in some embodiments, may alternatively change polarity. The magnetic field may stimulate muscles and may be used to provide one or more of the followings: relaxation of muscle spasms, prevention or retardation of disuse atrophy, increasing local blood circulation, muscle re-education, immediate post-surgical stimulation of calf muscles to prevent venous thrombosis, maintaining or increasing range of motion, etc.

With further reference to FIG. 1, the magnetic muscle stimulator 100 may include a novel cooling system that may cool the coil 140 by passing water from one or more water passages (or microchannels) through the interior of the coil 140. Using water, instead of air or oil, in the cooling system provides the technical advantage of the water being a more efficient cooling agent and helping the fat tissues of the person who is receiving therapy to get frozen while the treatment being safe and comfortable.

The use of water, instead of air provides the additional technical advantage of eliminating the need for air vents on the magnetic applicator 110 housing. A ventless magnetic applicator 110 provides the technical advantage of not emitting hot air out of the magnetic applicator 110 housing that may cause burn or discomfort to an operator or a person (e.g., a patient) who is using the magnetic muscle stimulator 100.

The use of a water passage inside the coil 140 provides the additional technical advantage of better controlling the temperature inside the coil. The use of a microchannel as the water passage inside the coil 140 provides the further technical advantage of more efficient heat transfer, because of a high surface-area to volume ratio. The use of the water cooling system provides the further technical advantage of allowing the use of a larger high power pulse generator 105, with a larger energy output and a higher working frequency, comparing to the existing magnetic muscle stimulators.

The water tank 120 may be filled by water through a water intake port, such as, the water injection port 121. The water may be supplied, for example, manually or through a cold tap water pipe. The water, in some embodiments, may be distilled or deionized water and may be supplied through a filter or from a distilled or deionized water storage (e.g., a bottle, a container, a tank, etc.).

The water level sensor 122 may measure the water level inside the water tank 120 and may send the measurements to the processor 130. The processor 130 may display a warning message on the display 135 when the water level in the water tank 120 is below a threshold. The water tank 120 may include a water overflow port 123 to prevent the water from overflowing from the water tank 120.

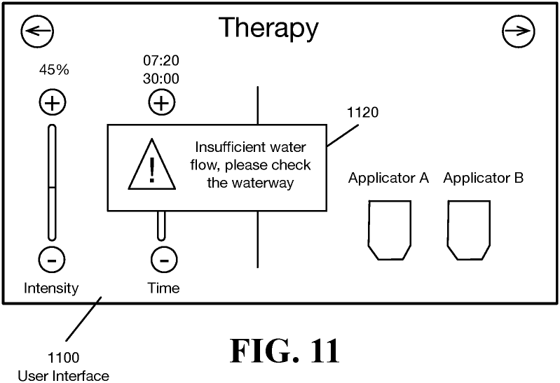

The cold water from the water tank 120 may be transferred by a pipe 116 to the pump 115. The pump 115 may pump the cold water through a separate pipe 117 to each magnetic applicator 110 (only one pipe 117 and one magnetic applicator 110 are shown for clarity). One or more water flow sensors 128 (only one is shown for clarity) may measure the water flow in different pipes and may send the measurements to the processor 130. The processor 130 may display the measurements, may display the status of the water flow (e.g., normal, slow, fast, etc.), and/or may stop the magnetic muscle stimulator 100 if the water flow measurements are above a maximum threshold or below a minimum threshold. An example of a message displayed by the processor 130 when the water flow is less than a minimum threshold is described below with reference to FIG. 11. The cold water supplied by the cold water pipe 117 may go through one or more microchannels inside the coil 140 and the heated water may exit the magnetic applicator 110 through to hot water pipe 119.

FIG. 2 is a top perspective view of a cross section of a magnetic applicator 110, according to various aspects of the present disclosure. With reference to FIG. 2, a portion of the housing 290 of the magnetic applicator 110 is removed to show the components inside the housing 290.

The coil 140 in the depicted embodiment is a 10 circle coil metallic coil. The coil 140, in some embodiments, may be permanently attached to the housing 290 by an adhesive material 280, such as, for example, and without limitations, glue. The high power electricity is delivered to the two ends of the coil through the wires 210 and 215. The cold water is supplied through the cold water pipe 117. The cold water goes through one or more microchannels inside the coil 140, gets heated up, and the hot water is returned to the pump 115 (FIG. 1) through the hot water pipe 119. The wires 210 and 215, and the water pipes 117 and 110, may be secured to the coil 140 by rubber or other insulating material 250.

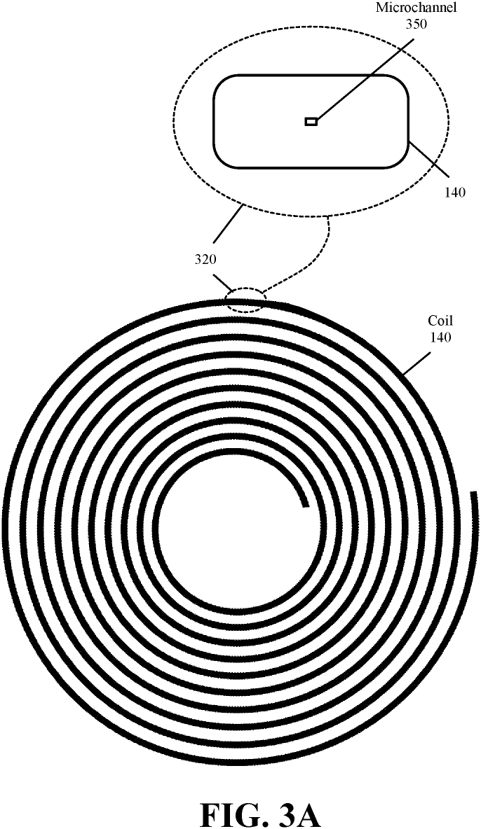

FIGS. 3A and 3B illustrate cross sections of coils 140 of magnetic applicators that include a microchannel for passing cooling water through, according to various aspects of the present embodiments. With reference to FIGS. 3A and 3B, the coil 140 may be similar to the coil 140 of FIGS. 1 and 2. The expanded views 320 and 330 show the cross section of the coil 140. The coil 140 may include a passage or microchannel 350 that may be used to pass the cooling water through the interior of the coil 140. The cold water may enter the microchannel through the cold water pipe 117 (FIGS. 1 and 2) and may exit through the hot water pipe 119 (FIGS. 1 and 2).

It should be noted that the coil 140, in different embodiments, may have different cross section shapes, such as rectangular, rectangular with curved corners, circular, ellipsoidal, or an arbitrary shape. In some embodiments, the cross section of the microchannel 350 may span across a large portion (e.g., 25% or more) of the cross section of the coil 140. The microchannel 350, in some embodiments, may have a height 380 of less than 3 mm. The microchannel 350, in some embodiments, may have a height 380 of less than 200 micrometer (um). The microchannel 350, in different embodiments, may have a width 390 of between 1 to 20 mm.

The term microchannel is used herein to refer to a fluid channel that has a height of less than 3 millimeter (mm) and preferably less than 0.2 mm. Although the term microchannel is used in describing several example embodiments, it should be noted that the water passage 350 inside the coil, in some embodiments, may have a height of more than 3 mm. Since the water passage 350 is inside the coil 140, the height 380 of the water passage 350 is only limited by the height (i.e., the thickness) 385 of the coil 140, as shown in FIG. 3B.

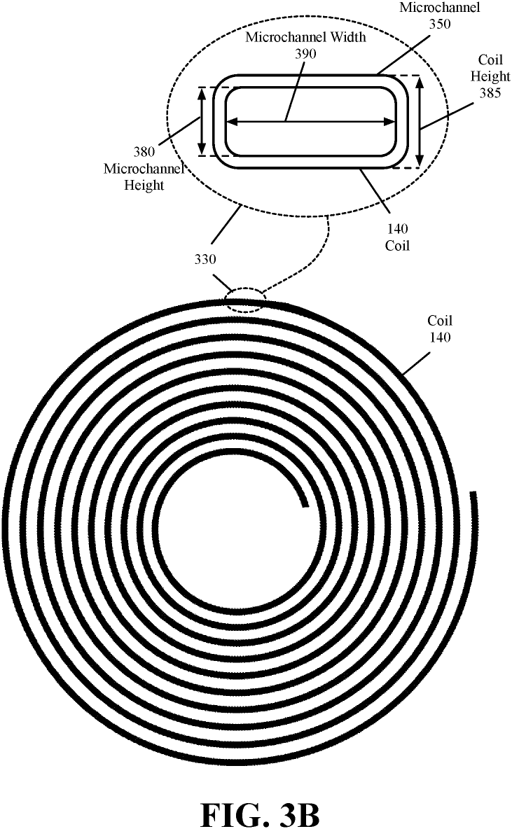

FIG. 4 illustrates a cross section of a coil 140 of a magnetic applicator that includes several microchannels for passing cooling water through, according to various aspects of the present embodiments. With reference to FIG. 4, the coil 140 may be similar to the coil 140 of FIGS. 1 and 2. The expanded view 420 shows the cross section of the coil 140. The coil 140 may include several passages or microchannels 450 that may be used to pass the cooling water through the coil 140. The cold water may enter the microchannels 450 through the cold water pipe 117 (FIGS. 1 and 2) and may exit through the hot water pipe 119 (FIGS. 1 and 2).

The microchannel 350 of FIGS. 3A-3B and the microchannel 450 of FIG. 4 provide the technical advantage of allowing the cooling water to pass through the interior of the coil 140, allowing a more effective way of cooling the coil 140 than passing a fluid, such as air or liquid, through the outside of the coil 140.

Referring back to FIG. 1, the pump 115 may pull the hot water from the coil 140 through the hot water pipe 119. The hot water pipe 119, in some embodiments, may pass from the vicinity of one or more fans 125 that may cool the hot water. The fan(s) 125, in different embodiments, may be positioned next to the pump 115, between the pump 115 and the coil 140, and/or between the pump 115 and the water tank 120. The hot water that is cooled by the fan(s) 125 is returned by the pump 115 into the water tank 120 through the return water pipe 118.

The water cooling system of the magnetic muscle stimulator 100 may be a closed loop system in which the water may circulate between the water tank 120 and the coil 140. Additional water may only be added to the system (e.g., through the water injection port 121) if the water is lost, for example, through evaporation. The water may be removed from the water cooling system through the drainage port 129 (e.g., to clean or maintain the system). The drainage port 129, in some embodiments, may include a valve that may open to start draining, when a hose is inserted into the drainage port 129. The water may also be removed through the water overflow port 123 if the water level in the water tank 120 exceeds a maximum threshold.

The magnetic muscle stimulator 100 may include one or more temperature sensors that may measure the temperature of the water and/or air at different locations and may send the measurements to the processor 130. For example, the magnetic muscle stimulator 100 may include a water temperature sensor 143 inside the water tank 120 to measure the temperature of the water inside the water tank 120. The magnetic muscle stimulator 100 may include a temperature sensor 142 inside each magnetic applicator 120 to measure the temperature of the air inside the magnetic applicator 120. The magnetic muscle stimulator 100 may include one or more temperature sensors 144 (only one temperature sensor is shown for clarity) that may measure the temperature of the water that passes through one or more of the pipes 116-119. The processor 130 may display the temperature measurements and/or may display warning messages if the temperatures exceed one or more thresholds.

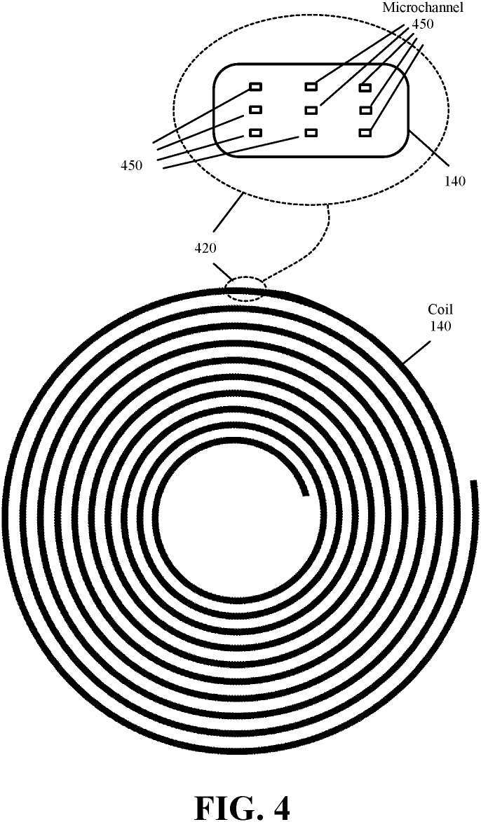

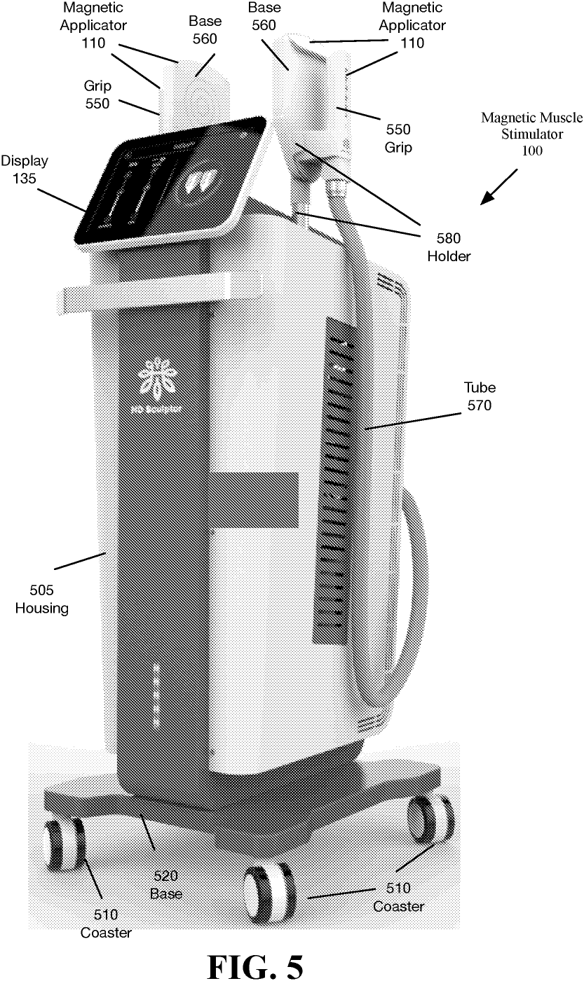

The magnetic muscle stimulator 100, in some embodiments, may be a portable device. FIG. 5 is a side perspective view of a magnetic muscle stimulator, according to various aspects of the present disclosure. FIG. 6 is a back perspective view of a magnetic muscle stimulator, according to various aspects of the present disclosure.

With reference to FIGS. 5 and 6, the magnetic muscle stimulator 100 may include a housing 505, a base 520, and several coasters 510. The coasters 510 may facilitate moving the magnetic muscle stimulator 100. The magnetic muscle stimulator 100 may include a power connector 610 (e.g., to connect the magnetic muscle stimulator 100 to an AC outlet) and a power switch 610 to turn on or off the electricity to the magnetic muscle stimulator 100.

The magnetic muscle stimulator 100 may include a water injection port 121, a water overflow port 123, and a drainage port 129. The magnetic muscle stimulator’s housing 505 may include one or more ventilation grids 655 to allow the air to circulate into the housing 505 (e.g., to circulate the air that goes through the fan(s) 125 of FIG. 1). It should be noted that the ventilation grids 655 are on the housing 505 of the muscle stimulator 100 and not on the housing 290 (FIG. 2) of the magnetic applicators 110, as the magnetic applicators 110 of the present embodiments are ventless and are not cooled by air.

In the depicted embodiments of FIGS. 5 and 6, the magnetic muscle stimulator 100 includes two magnetic applicators 110. Each magnetic applicator 110 may include a grip 550 (for holding the magnetic applicator by e.g., the operator), a base 560 (for contacting the body of the person who is receiving therapy), and a tube 570 (for providing electricity and water to the magnetic applicator). Each magnetic applicator 110 may be connected to, or disconnected from, the housing 505 of the magnetic muscle stimulator 100 by a connector 650 (FIG. 6) to a matching connector 670 on the magnetic muscle stimulator 100 housing 505.

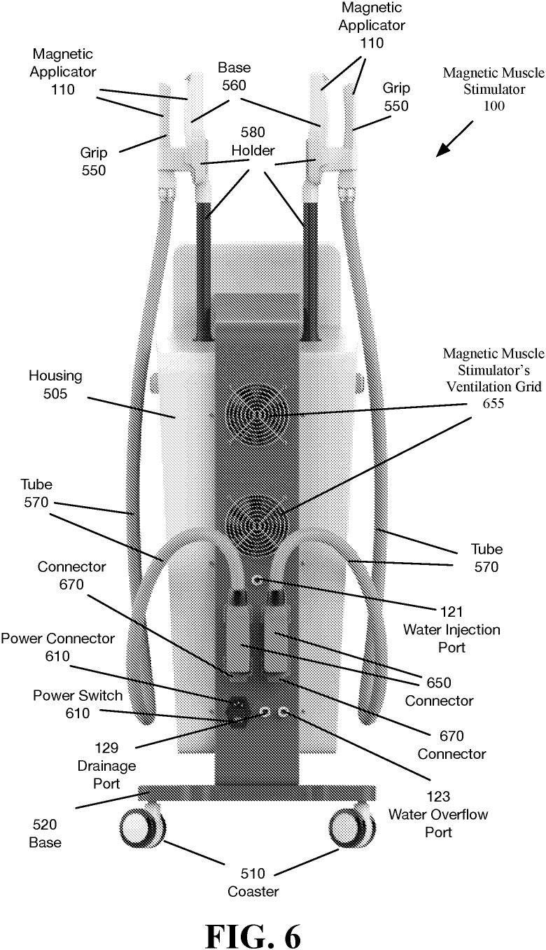

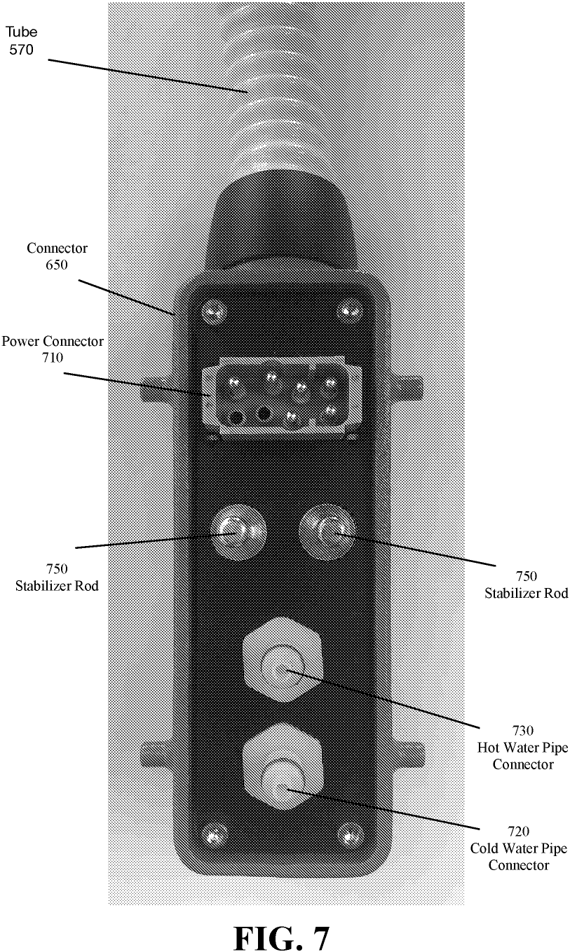

FIG. 7 is a front perspective view of a magnetic applicator connector 650 that connects to a matching connector on the housing of the magnetic muscle stimulator, according to various aspects of the present disclosure. FIG. 8 is a front perspective view of a connector 670 on the housing of the magnetic muscle stimulator 100 that connects to a matching connector on a magnetic applicator connector, according to various aspects of the present disclosure.

With reference to FIGS. 7-8, the magnetic applicator connector 650 may include a power connector 710 that may fit into the power connector 810 of the connector 670 on the housing of the magnetic muscle stimulator. The cold water pipe connector 720 of the connector 650 may fit into the cold water pipe 119 (FIG. 8). The hot water connector 730 may fit into the hot water pipe 119.

The magnetic applicator connector 650 and the connector 670, in some embodiments, may provide a quick connect and disconnect mechanism. For example, the magnetic applicator connector 650 may include one or more stabilizer rods 750 that may fit into corresponding receptors 850 on the connector 670 to provide stability and to securely attach the magnetic applicator 110 to the housing 505 of the magnetic muscle stimulator 100. In alternative embodiments, the connector 670 may include one or more stabilizer rods that may fit into corresponding receptors on the magnetic applicator connector 650.

The connectors 650 and 670 (FIGS. 6-8) provide the technical advantage of allowing the magnetic applicators 110 to be easily attached and detached from the housing 505 without the need for any special tools. In contrast, the prior art magnetic applicators require special tools to attach to, and detach from, the magnetic muscle stimulator housing.

With reference to FIG. 1, the magnetic applicators 110 may, in some embodiments, include unique identification codes that may be readable by the processor 130. The processor 130 may store the total working time of each uniquely identified magnetic applicator 110. The magnetic applicators 110 may be turned on or off through the user interface displayed on the display 135. Whenever a magnetic applicator 110 is turned on, the processor 130 may keep track of the working time of the magnetic applicator 110 and may update the total working time of the magnetic applicator 110. Once a magnetic applicator 110 has been used for a threshold amount of time (e.g., and without limitations 500 hours, 600 hours, 700 hours, etc.) the processor 130 may prevent the magnetic applicator 110 from being used any further.

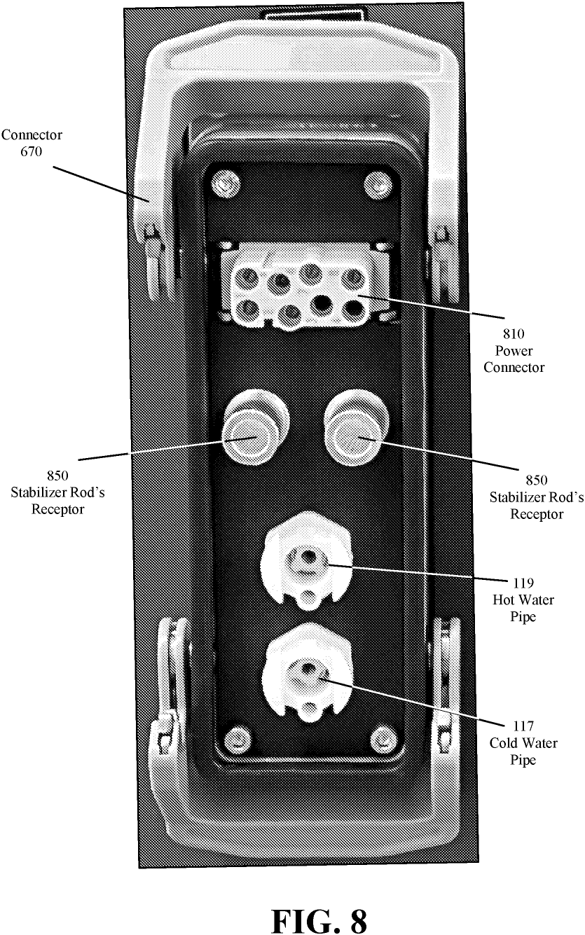

FIG. 9 is a schematic front view of the display 135 of a magnetic muscle stimulator with a user interface that indicates the working time of each magnetic applicator, according to various aspects of the present disclosure. With reference to FIG. 9, the processor 130 (FIG. 1) of the magnetic muscle stimulator 100 may display the user interface 900 on the display 135 of the magnetic muscle stimulator 100 when an option such as setting is selected. The user interface 900 may provide different options to set the volume 901, to set the screen brightness 902, to display the software version 903, and to calibrate the applicators 904. The user interface 900 may display the total working time 910 and the maximum allowable working time 920 of each magnetic applicator (e.g., in minutes).

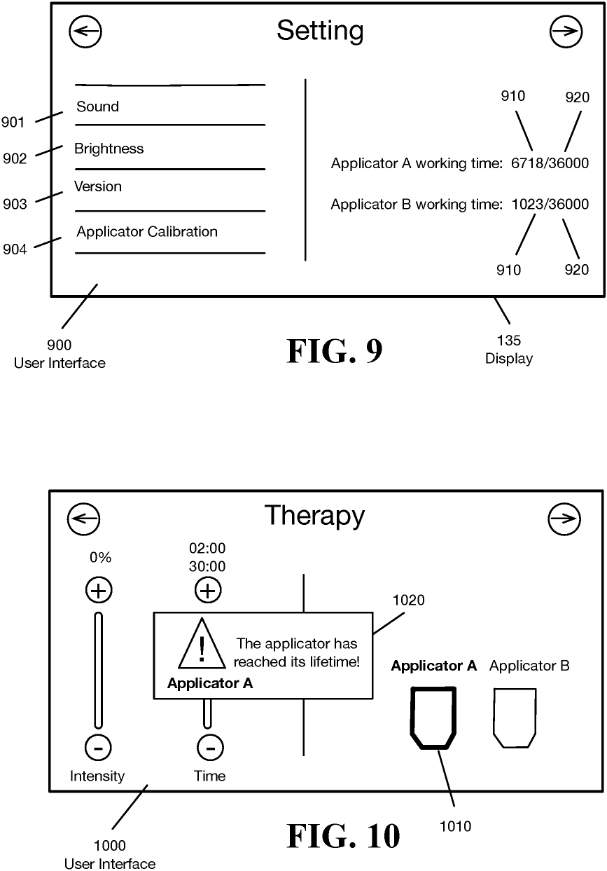

FIG. 10 is a schematic front view of the display of a magnetic muscle stimulator with a user interface that provides a warning when a magnetic applicator exceeds the maximum working time, according to various aspects of the present disclosure. As shown in FIG. 10, the processor 130 has determined that the magnetic applicator A 1010 has exceeded the maximum allowable working time.

The user interface 1000 may display a warning message 1020 that may identify the applicator that has reached its lifetime. The processor 130 may prevent the identified applicator 1010 to be used any further. The connector mechanism 650 of FIGS. 6-8 allows the quick removal of the magnetic applicator 1010 and replacing it with a working magnetic applicator.

The processor 130 (FIG. 1) may monitor the measurements received from different sensors and may generate warning messages and/or may stop the magnetic muscle stimulator from operating when the measurements are not within tolerance levels. For example, the processor 130 may receive water flow measurements from the water flow sensor(s) 128 and may compare the water flow measurements with tolerable levels and may generate a message when the water flow is either below a minimum threshold or above a maximum threshold.

FIG. 11 is a schematic front view of the display of a magnetic muscle stimulator with a user interface that provides a warning when the magnetic muscle stimulator has insufficient water flow, according to various aspects of the present disclosure. In the example of FIG. 11, the processor 130 may have determined that the water flow measurements are below a minimum threshold.

As shown in FIG. 11, the user interface 1100 may display a warning message 1120 indicating that there is insufficient water flow, and may instruct the operator to check the waterway. The processor 130, in some embodiments, may prevent the magnetic muscle stimulator 100 from operating. For example, the processor 130 may turn off the power to the magnetic applicator(s) and may prevent the magnetic applicator(s) from operating until the water flow measurement are within tolerable thresholds.

Some of the above-described features and applications are implemented as software processes that are specified as a set of instructions recorded on a computer readable storage medium (also referred to as computer readable medium). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

In this specification, the term “software” is meant to include firmware residing in read-only memory or applications stored in magnetic storage, which may be read into memory for processing by a processor. Also, in some embodiments, multiple software inventions may be implemented as sub-parts of a larger program while remaining distinct software inventions. In some embodiments, multiple software inventions may also be implemented as separate programs. Finally, any combination of separate programs that together implement a software invention described here is within the scope of the invention. In some embodiments, the software programs, when installed to operate on one or more electronic systems, define one or more specific machine implementations that execute and perform the operations of the software programs.

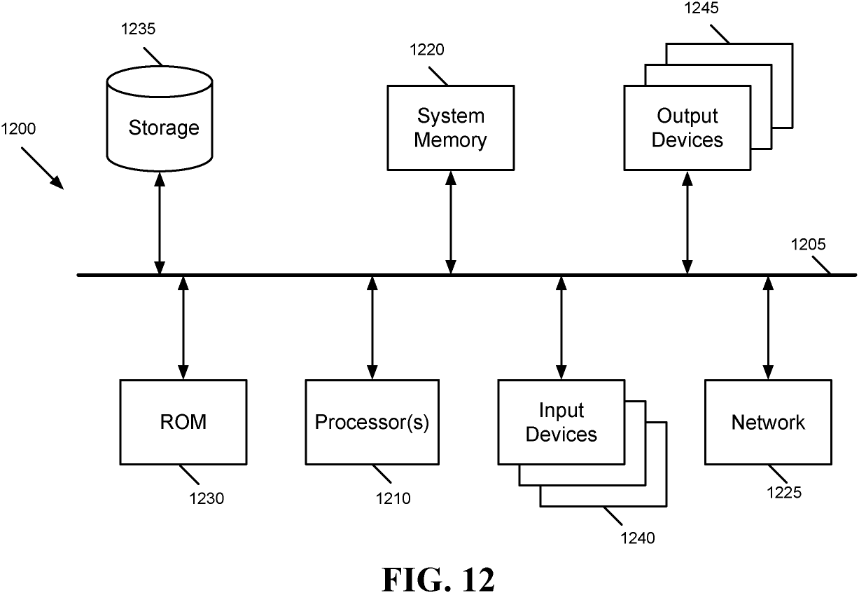

FIG. 12 conceptually illustrates an electronic system 1200 with which some embodiments of the invention (e.g., the magnetic muscle stimulator 100, described above) are implemented. The electronic system 1200 may be used to execute any of the control, virtualization, or operating system applications described above. The electronic system 1200 may be a computer or any other sort of computing device. Such an electronic system includes various types of computer readable media and interfaces for various other types of computer readable media. The electronic system 1200 includes a bus 1205, processing unit(s) 1210, a system memory 1220, a read-only memory (ROM) 1230, a permanent storage device 1235, input devices 1240, and output devices 1245.

The bus 1205 collectively represents all system, peripheral, and chipset buses that communicatively connect the numerous internal devices of the electronic system 1200. For instance, the bus 1205 communicatively connects the processing unit(s) 1210 with the read-only memory 1230, the system memory 1220, and the permanent storage device 1235.

From these various memory units, the processing unit(s) 1210 retrieve(s) instructions to execute and data to process in order to execute the processes of the invention. The processing unit(s) may be a single processor or a multi-core processor in different embodiments.

The read-only-memory 1230 stores static data and instructions that are needed by the processing unit(s) 1210 and other modules of the electronic system. The permanent storage device 1235, on the other hand, is a read-and-write memory device. This device is a non-volatile memory unit that stores instructions and data even when the electronic system 1200 is off. Some embodiments of the invention use a mass-storage device (such as a magnetic or optical disk and its corresponding disk drive) as the permanent storage device 1235.

Other embodiments use a removable storage device (such as a flash drive, memory cards, etc.) as the permanent storage device. Like the permanent storage device 1235, the system memory 1220 is a read-and-write memory device. However, unlike storage device 1235, the system memory is a volatile read-and-write memory, such as random access memory. The system memory stores some of the instructions and data that the processor needs at runtime. In some embodiments, the invention’s processes are stored in the system memory 1220, the permanent storage device 1235, and/or the read-only memory 1230. From these various memory units, the processing unit(s) 1210 retrieve instructions to execute and data to process in order to execute the processes of some embodiments.

The bus 1205 also connects to the input and output devices 1240 and 1245. The input devices enable the user to communicate information and select commands to the electronic system. The input devices 1240 may include alphanumeric keyboards and pointing devices (also called “cursor control devices”). The input devices 1240, in some embodiments, may include cameras, sensors, microphones, near field communication (NFC) readers, and/or radio-frequency identification (RFID) readers. The input devices 1240, in some embodiments, may include pushbutton, switches, and/or knobs. The output devices 1245 may include printers, speakers, light sources (e.g., flashlights), and display devices, such as cathode ray tubes (CRT), liquid-crystal displays (LCD), light-emitting diode (LED) displays. Some embodiments may include devices, such as a touchscreen, that function as both input and output devices. The output devices 1245, in some embodiments, may display images generated and/or received by the electronic system.

Finally, as shown in FIG. 12, bus 1205 also couples electronic system 1200 to a network 1225 through a network adapter (not shown). In this manner, the computer may be a part of a network of computers (such as a local area network (“LAN”), a wide area network (“WAN”), an Intranet, or a network of networks, such as the Internet. Any or all components of electronic system 1200 may be used in conjunction with the invention.

Some embodiments include electronic components, such as microprocessors, storage, and memory, that store computer program instructions in a machine-readable or computer-readable medium (alternatively referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra-density optical discs, any other optical or magnetic media. The computer-readable media may store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some embodiments are performed by one or more integrated circuits, such as application specific integrated circuits (ASICs) or field programmable gate arrays (FPGAs). In some embodiments, such integrated circuits execute instructions that are stored on the circuit itself.

As used in this specification, the terms “computer”, “server”, “processor”, and “memory” all refer to electronic or other technological devices. These terms exclude people or groups of people. For the purposes of the specification, the terms display or displaying means displaying on an electronic device. As used in this specification, the terms “computer readable medium,” “computer readable media,” and “machine readable medium” are entirely restricted to tangible, non-transitory, physical objects that store information in a form that is readable by a computer. These terms exclude any wireless signals, wired download signals, and any other ephemeral or transitory signals.

In a first aspect, a magnetic muscle stimulator device is provided. The magnetic muscle stimulator device comprises a water tank configured to hold a quantity of water; a pump; a set of one or more fans; and a magnetic applicator comprising a coil. The coil generates a magnetic field in response to receiving an alternating current (AC). The coil comprises a water passage in the interior of the coil. The pump receives water from the water tank through a first water pipe and transfers the water into the water passage of the coil through a second water pipe. The water absorbs heat from the coil while passing through the water passage of the coil. The pump receives the water from the water passage of the coil through a third pipe and returns the water into the water tank through a fourth pipe. The water is cooled by the set of one or more fans while being returned from the water passage of the coil into the water tank.

In an embodiment of the first aspect, the magnetic applicator further comprises a housing encompassing the coil and the housing of the magnetic applicator does not comprise an air vent.

An embodiment of the first aspect further comprises: a water flow sensor configured to measure a flow of water through the pump, the first, second, third, and fourth pipes, and the water passage inside the coil; a processor; and a display. The processor is configured to: receive measurements from the water flow sensor; compare the measurements against a minimum threshold and a maximum threshold; and display a warning message on the display when the flow of water is below the minimum threshold or above the maximum threshold.

In another embodiment of the first aspect, the processor is further configured to disconnect the AC to the coil when the flow of water is below the minimum threshold or above the maximum threshold.

Another embodiment of the first aspect further comprises: a temperature sensor configured to measure a temperature of water in the water tank; a processor; and a display. The processor is configured to: receive measurements from the temperature sensor; compare the measurements against a threshold; and display a warning message on the display when the temperature of water in the water tank is above the maximum threshold.

Another embodiment of the first aspect further comprises: a temperature sensor configured to measure a temperature of one of the first, second, third, and fourth pipes; a processor; and a display. The processor is configured to: receive measurements from the temperature sensor; compare the measurements against a threshold; and display a warning message on the display when the temperature of water received from the temperature sensor is above the maximum threshold.

In another embodiment of the first aspect, the magnetic applicator further comprises a housing encompassing the coil. The magnetic muscle stimulator device further comprises: a temperature sensor configured to measure a temperature of air inside the housing of the magnetic applicator; a processor; and a display. The processor is configured to: receive measurements from the temperature sensor; compare the measurements against a threshold; and display a warning message on the display when the temperature of the air inside the magnetic applicator is above the maximum threshold.

Another embodiment of the first aspect further comprises a processor and a display. The processor is configured to: keep track of a total working time of the magnetic applicator; compare the total working time of the magnetic applicator with a threshold; display a warning message on the display when the total working time of the magnetic applicator exceeds threshold; and disconnect the AC to the coil when the total working time of the magnetic applicator exceeds threshold.

In another embodiment of the first aspect, the magnetic applicator is a first magnetic applicator. The magnetic muscle stimulator device further comprises a plurality of magnetic applicators including the first magnetic applicator, a processor, and a display. Each magnetic applicator comprises a unique identification code. The processor is configured to: read the identification code of each magnetic applicator that is connected to the muscle stimulator device; keep track of a total working time of each magnetic applicator using the corresponding identification code; compare the total working time of each magnetic applicator with a threshold; display a warning message on the display when the total working time of a magnetic applicator exceeds threshold; and disconnect the AC to the coil of the magnetic applicator when the total working time of the magnetic applicator exceeds threshold.

In another embodiment of the first aspect, the water tank comprises a water injection port for adding water to the water tank and a water overflow port configured to prevent the water from overflowing from the water tank.

In another embodiment of the first aspect, the water tank comprises a drainage port comprising a valve. The valve of the drainage port is configured to open when a hose is inserted into the drainage port.

Another embodiment of the first aspect further comprises a capacitor and a high power pulse generator. The high power pulse generator is configured to: receive an AC from an AC source external to the magnetic muscle stimulator device; generate high power pulses with a power ranging between 1,200 volt-amperes (VA) to 2,300 VA; and charge and discharge the capacitor by the high power pulses. The capacitor is configured to provide energy in the form AC to the coil.

In another embodiment of the first aspect, the water passage in the interior of the coil is a microchannel with a height of less than one millimeter.

In another embodiment of the first aspect, the water passage in the interior of the coil is a microchannel with a height of less than 200 micrometer.

In another embodiment of the first aspect, a cross section of the water passage in the interior of the coil covers at least 25 percent of a cross section of the coil.

In another embodiment of the first aspect, the magnetic applicator comprises a housing encompassing the coil. The magnetic muscle stimulator device further comprises a housing encompassing the water tank and the pump; a tube connected to the housing of the magnetic applicator. The magnetic applicator and the housing of the magnetic muscle stimulator device comprise a quick connect and disconnect mechanism comprising: a first connector connected to the tube; and a second connector connected to the housing of the magnetic muscle stimulator device. The first connector comprises one or more stabilizer rods. Each stabilizer rod of the first connector fits into a corresponding receptor on the second connector.

In another embodiment of the first aspect, the magnetic applicator comprises a housing encompassing the coil. The magnetic muscle stimulator device further comprises a housing encompassing the water tank and the pump; and a tube connected to the housing of the magnetic applicator. The magnetic applicator and the housing of the magnetic muscle stimulator device comprise a quick connect and disconnect mechanism comprises a first connector connected to the tube and a second connector connected to the housing of the magnetic muscle stimulator device. The second connector comprises one or more stabilizer rods and each stabilizer rod of the second connector fits into a corresponding receptor on the first connector.

In another embodiment of the first aspect, the water passage in the interior of the coil is a first water passage in the interior of the coil, the coil further comprises a set of one or more water passages in the interior of the coil other than the first water passage, the pump transfers the water into the set of one or more water passages through the second water pipe; the water absorbs heat from the coil while passing through the set of one or more water passages; and the pump receives the water from the set of one or more water passages through the third pipe.

While the invention has been described with reference to numerous specific details, one of ordinary skill in the art will recognize that the invention may be embodied in other specific forms without departing from the spirit of the invention. In addition, a number of the figures conceptually illustrate processes. The specific operations of these processes may not be performed in the exact order shown and described. The specific operations may not be performed in one continuous series of operations, and different specific operations may be performed in different embodiments. Furthermore, the process could be implemented using several sub-processes, or as part of a larger macro process.

The above description presents the best mode contemplated for carrying out the present embodiments, and of the manner and process of practicing them, in such full, clear, concise, and exact terms as to enable any person skilled in the art to which they pertain to practice these embodiments. The present embodiments are, however, susceptible to modifications and alternate constructions from those discussed above that are fully equivalent. Consequently, the present invention is not limited to the particular embodiments disclosed. On the contrary, the present invention covers all modifications and alternate constructions coming within the spirit and scope of the present disclosure. For example, the steps in the processes described herein need not be performed in the same order as they have been presented and may be performed in any order(s). Further, steps that have been presented as being performed separately may in alternative embodiments be performed concurrently. Likewise, steps that have been presented as being performed concurrently may in alternative embodiments be performed separately.

What is claimed is:

1. A magnetic muscle stimulator device, comprising:
a water tank configured to hold a quantity of water;
a pump;
a set of one or more fans; and
a magnetic applicator comprising a coil;
wherein the coil generates a magnetic field in response to receiving an alternating current (AC);
wherein the coil comprises a water passage in an interior of the coil;
wherein the pump receives water from the water tank through a first water pipe and transfers the water into the water passage of the coil through a second water pipe;
wherein the water absorbs heat from the coil while passing through the water passage of the coil;
wherein the pump receives the water from the water passage of the coil through a third pipe and returns the water into the water tank through a fourth pipe; and
wherein the water is cooled by the set of one or more fans while being returned from the water passage of the coil into the water tank.

2. The magnetic muscle stimulator device of claim 1, wherein the magnetic applicator further comprises a housing encompassing the coil, and wherein the housing of the magnetic applicator does not comprise an air vent.

3. The magnetic muscle stimulator device of claim 1, further comprising:
a water flow sensor configured to measure a flow of water through the pump, the first, second, third, and fourth pipes, and the water passage inside the coil;
a processor; and
a display;
wherein the processor is configured to:
receive measurements from the water flow sensor;
compare the measurements against a minimum threshold and a maximum threshold; and
display a warning message on the display when the flow of water is below the minimum threshold or above the maximum threshold.

4. The magnetic muscle stimulator device of claim 3, wherein the processor is further configured to disconnect the AC to the coil when the flow of water is below the minimum threshold or above the maximum threshold.

5. The magnetic muscle stimulator device of claim 1, further comprising:
a temperature sensor configured to measure a temperature of water in the water tank;
a processor; and
a display;
wherein the processor is configured to:
receive measurements from the temperature sensor;
compare the measurements against a threshold; and
display a warning message on the display when the temperature of water in the water tank is above the maximum threshold.

6. The magnetic muscle stimulator device of claim 1, further comprising:
a temperature sensor configured to measure a temperature of one of the first, second, third, and fourth pipes;
a processor; and
a display;
wherein the processor is configured to:
receive measurements from the temperature sensor;
compare the measurements against a threshold; and
display a warning message on the display when the temperature of water received from the temperature sensor is above the maximum threshold.

7. The magnetic muscle stimulator device of claim 1, wherein the magnetic applicator further comprises a housing encompassing the coil, the magnetic muscle stimulator device further comprising:
a temperature sensor configured to measure a temperature of air inside the housing of the magnetic applicator;
a processor; and
a display;
wherein the processor is configured to:
receive measurements from the temperature sensor;
compare the measurements against a threshold; and
display a warning message on the display when the temperature of the air inside the magnetic applicator is above the maximum threshold.

8. The magnetic muscle stimulator device of claim 1 further comprising:
a processor; and
a display;
wherein the processor is configured to:
keep track of a total working time of the magnetic applicator;
compare the total working time of the magnetic applicator with a threshold;
display a warning message on the display when the total working time of the magnetic applicator exceeds threshold; and
disconnect the AC to the coil when the total working time of the magnetic applicator exceeds threshold.

9. The magnetic muscle stimulator device of claim 1, wherein the magnetic applicator is a first magnetic applicator, the magnetic muscle stimulator device further comprising:
a plurality of magnetic applicators including the first magnetic applicator;
a processor; and
a display;
wherein each magnetic applicator comprises a unique identification code;
wherein the processor is configured to:
read the identification code of each magnetic applicator that is connected to the muscle stimulator device;
keep track of a total working time of each magnetic applicator using the corresponding identification code;
compare the total working time of each magnetic applicator with a threshold;
display a warning message on the display when the total working time of a magnetic applicator exceeds threshold; and
disconnect the AC to the coil of the magnetic applicator when the total working time of the magnetic applicator exceeds threshold.

10. The magnetic muscle stimulator device of claim 1, wherein the water tank comprises:
a water injection port for adding water to the water tank; and
a water overflow port configured to prevent the water from overflowing from the water tank.

11. The magnetic muscle stimulator device of claim 1, wherein the water tank comprises:
a drainage port comprising a valve;
wherein the valve of the drainage port is configured to open when a hose is inserted into the drainage port.

12. The magnetic muscle stimulator device of claim 1, further comprising:
a capacitor; and
a high power pulse generator configured to:
receive an AC from an AC source external to the magnetic muscle stimulator device;
generate high power pulses with a power ranging between 1,200 volt-amperes (VA) to 2,300 VA; and
charge and discharge the capacitor by the high power pulses;
wherein the capacitor is configured to provide energy in the form AC to the coil.

13. The magnetic muscle stimulator device of claim 1, wherein the water passage in the interior of the coil is a microchannel with a height of less than one millimeter.

14. The magnetic muscle stimulator device of claim 1, wherein the water passage in the interior of the coil is a microchannel with a height of less than 200 micrometer.

15. The magnetic muscle stimulator device of claim 1, wherein a cross section of the water passage in the interior of the coil covers at least 25 percent of a cross section of the coil.

16. The magnetic muscle stimulator device of claim 1, wherein the magnetic applicator comprises a housing encompassing the coil, the magnetic muscle stimulator device further comprising:
a housing encompassing the water tank and the pump; and
a tube connected to the housing of the magnetic applicator;
wherein the magnetic applicator and the housing of the magnetic muscle stimulator device comprise a quick connect and disconnect mechanism, comprising:
a first connector connected to the tube; and
a second connector connected to the housing of the magnetic muscle stimulator device;
wherein the first connector comprises one or more stabilizer rods; and
wherein each stabilizer rod of the first connector fits into a corresponding receptor on the second connector.

17. The magnetic muscle stimulator device of claim 1, wherein the magnetic applicator comprises a housing encompassing the coil, the magnetic muscle stimulator device further comprising:
a housing encompassing the water tank and the pump; and
a tube connected to the housing of the magnetic applicator;
wherein the magnetic applicator and the housing of the magnetic muscle stimulator device comprise a quick connect and disconnect mechanism comprising:
a first connector connected to the tube; and
a second connector connected to the housing of the magnetic muscle stimulator device;
wherein the second connector comprises one or more stabilizer rods; and
wherein each stabilizer rod of the second connector fits into a corresponding receptor on the first connector.

18. The magnetic muscle stimulator device of claim 1,
wherein the water passage in the interior of the coil is a first water passage in the interior of the coil,
wherein the coil further comprises a set of one or more water passages in the interior of the coil other than the first water passage,
wherein the pump transfers the water into the set of one or more water passages through the second water pipe,
wherein the water absorbs heat from the coil while passing through the set of one or more water passages, and
wherein the pump receives the water from the set of one or more water passages through the third pipe.
